(19) United States
(12) Patent Application Publication
(10) Pub. No.: US 2006/0128034 A1
(43) Pub. Date: Jun. 15, 2006

(54) Diagnostic test using gated measurement of fluorescence from quantum dots

(76) Inventors: Patrick T. Petruno, San Jose, CA (US); Daniel B. Roitman, Menlo Park, CA (US); Rong Zhou, Sunnyvale, CA (US); John F. Petrilla, Palo Alto, CA (US); Marcel P. Bruchez, Belmont, CA (US); Andrew R. Watson, Belmont, CA (US)

Correspondence Address: AGILENT TECHNOLOGIES, INC.;Legal Department, DL 429, Intellectual Property Administration, P.O Box 7599, Loveland, CO, 80537-0599, US

(21) Appl. No.: 11/008,912

(22) Filed: Dec. 10, 2004

(51) Int. Cl. G01N 33/551; C12M 1/34

(52) U.S. Cl. 436/524; 435/287.2; 977/920

(57) ABSTRACT

A rapid diagnostic test system or process uses a gated measurement of the fluorescent light from quantum dots after shutting off an illuminating light source. A delay between shutting off the illumination and measuring allows background fluorescence from substances other than the quantum dots to drop significantly when compared to the intensity of the fluorescence from the quantum dots. Using quantum dots permits high measurement repetition rates and good extinction of background fluorescence.

### BACKGROUND

Rapid diagnostic test kits are currently available for testing for a wide variety of medical and environmental conditions. Commonly, such test kits employ an analyte-specific binding assay to detect or measure a specific environmentally or biologically relevant compound such as a hormone, a metabolite, a toxin, or a pathogen-derived antigen.

A convenient structure for performing a binding assay is a “lateral flow” strip such as test strip 100 illustrated in FIG. 1. Test strip 100 includes several “zones” that are arranged along a flow path of a sample. In particular, test strip 100 includes a sample receiving zone 110, a labeling zone 120, a capture or detection zone 130, and an absorbent zone or sink 140. Zones 110, 120, 130, and 140, which can be attached to a common backing 150, are generally made of a material such as chemically treated nitrocellulose that allows fluid flow by capillary action.

An advantage of test strip 100 and of a lateral flow immunoassay generally is the ease of the testing procedure and the rapid availability of test results. In particular, a user simply applies a liquid sample such as blood, urine, or saliva to sample receiving zone 110. Capillary action then draws the liquid sample downstream into labeling zone 120, which contains a substance for indirect labeling of a target analyte. For medical testing, the labeling substances are generally immunoglobulin with attached dye molecules but alternatively may be a non-immunoglobulin labeled compound that specifically binds the target analyte.

The sample flows from labeling zone 120 into capture zone 130 where the sample contacts a test region or stripe 132 containing an immobilized compound capable of specifically binding the labeled target analyte or a complex that the analyte and labeling substance form. As a specific example, analyte-specific immunoglobulins can be immobilized in capture zone 130. Labeled target analytes bind the immobilized immunoglobulins, so that test stripe 132 retains the labeled analytes. The presence of the labeled analyte in the sample generally results in a visually detectable coloring in test stripe 132 that appears within minutes of starting the test.

A control stripe 134 in capture zone 130 is useful for indicating that a procedure has been performed. Control stripe 134 is downstream of test stripe 132 and operates to bind and retain the labeling substance. Visible coloring of control stripe 134 indicates the presence of the labeling substance resulting from the liquid sample flowing through capture zone 130. When the target analyte is not present in the sample, test stripe 132 shows no visible coloring, but the accumulation of the labeling substance in control stripe 134 indicates that the sample has flown through capture zone 130. Absorbent zone 140 then captures any excess sample.

One problem with these immunoassay procedures is the difficulty in providing quantitative measurements. In particular, a quantitative measurement may require determining the number of labeled complexes bound in test stripe 132. Measuring equipment for such determinations can be expensive and is vulnerable to contamination since capture zone 120, which contains the sample, is generally exposed for measurement. Further, the intensity of dyes used in the test typically degrade very rapidly (e.g., within minutes or hours) when exposed to light, so that quantitative measurements based on the intensity of color must somehow account for dye degradation. On the other hand, a home user of a single-use rapid diagnostic test kit may have difficulty interpreting a test result from the color or shade of test stripe 132, particularly since dye intensity declines within minutes.

Another testing technology, which is generally performed in laboratories, simultaneously subjects a sample to a panel of tests. For this type of testing, portions of a sample can be applied to separate test solutions. Each test solution generally contains a labeled compound that specifically binds a target analyte associated with the test being performed. Conventionally, the tests are separate because the labeled compounds that bind different target analytes are typically difficult to distinguish if combined in the same solution.

U.S. Pat. No. 6,630,307, entitled “Method of Detecting an Analyte in a Sample Using Semiconductor Nanocrystals as a Detectable Label,” describes a process that labels binding compounds for different target analytes with different types of semiconductor nanocrystals or quantum dots. The different types of nanocrystals when exposed to a suitable wavelength of light fluoresce to produce light of different wavelengths. Accordingly, binding compounds labeled with different combinations of quantum dots can be distinguished by spectral analysis of the fluorescent light emitted from the quantum dots.

### SUMMARY

In accordance with an aspect of the invention, a rapid diagnostic test system employs a labeling substance that attaches a quantum dot to a target analyte. When a detection zone that binds the labeled target analyte is illuminated, the quantum dots in the labeling substance fluoresce and emit a relatively bright light with a stable wavelength. The intensity of the fluorescent light from the quantum dots generally depends on and indicates the number of target analytes that are bound in the detection zone of the test system. A measurement of the light emitted at the wavelength associated with the quantum dots can thus provide a quantitative measurement of the concentration of a target analyte. In accordance with a further aspect of the invention, the illumination that causes the quantum dots to fluoresce stops before the measurement of the fluorescent light. The delay between stopping the illumination and measuring light intensity can be selected according to the persistence of fluorescence from the quantum dots and other materials in the test system. Fluorescence from other materials (e.g., typical organic materials) in the test system generally declines more rapidly than does the fluorescence from the quantum dots. Accordingly, delaying measurement after shutting off the source of illumination can provide a high signal-to-noise ratio, accurate quantitative measurements, and high sensitivity.

In accordance with a further aspect of the invention, a decay time of the fluorescence of quantum dots, which is long enough that a gated measurement provides a high signal to noise ratio, is sufficiently short for rapid repetition of gated measurements. The repetitions of the gated measurements provide statistics for better measurement accuracy without requiring an unacceptably long measurement time.

One specific embodiment of the invention is a rapid diagnostic test system including a light source, a photodetector, and a control system. The light source illuminates a medium such as a lateral-flow strip containing a sample under test and a labeling substance that binds a quantum dot to a target analyte. The photodetector measures light from a test area of the medium. The control system is coupled to the light source and the photodetector and executes a measurement processes including processing a measurement signal from the photodetector that indicates a light intensity after the light source has been off for a time.

Another specific embodiment of the invention is a process for rapid diagnostic testing. The process includes: applying a sample to a medium containing a labeling substance that binds a quantum dot to a target analyte; illuminating a portion of the medium with light capable of causing the quantum dot to fluoresce; stopping the illumination of the portion of the medium; measuring light from the portion of the medium after the illumination remains stopped for a delay time; and determining a test result from the measuring of the light.

### BRIEF DESCRIPTION OF THE DRAWINGS

FIG. 1 shows a conventional test strip for an analyte-specific binding assay.

FIG. 2 shows a cross-sectional view of an optoelectronic rapid diagnostic test system in accordance with an embodiment of the invention.

FIG. 3 illustrates the drop in the intensity of fluorescent light after a source driving the fluorescence is turned off.

FIG. 4 is a flow diagram a rapid diagnostic test method using a gated measurement of the fluorescent light from a label substance containing quantum dots.

FIG. 5 illustrates an embodiment of the invention using an imaging system to measure the intensity of fluorescent light from quantum dots.

FIG. 6 illustrates a test system in accordance with an embodiment of the invention using a diffractive optical substrate for focusing and filtering.

FIG. 7 illustrates a test system in accordance with an embodiment of the invention using refractive lenses and thin-film color filters for optical signals.

Use of the same reference symbols in different figures indicates similar or identical items.

### DETAILED DESCRIPTION

In accordance with an aspect of the invention, a rapid diagnostic test system employs quantum dots as labels for a target analyte and gated measurements of the fluorescent light from quantum dots for generation of quantitative or qualitative test results. The test system can include a light source that illuminates a test area with light of the proper wavelength to cause fluorescence of the quantum dots, a photodetector such as a photodiode or a sensor array that measures the resulting fluorescent light to detect the target analyte, and a control system that shuts off the light source and waits a prescribed interval before using the photodetector or sensor array for measurement of the fluorescent light from the quantum dots.

FIG. 2 shows a cross-section of a test system 200 in accordance with an embodiment of the invention where an optoelectronic device reads a test result. In various embodiments of the invention, system 200 can test for any desired medical or environmental condition or substance including but not limited to glucose, pregnancy, infectious diseases, cholesterol, cardiac markers, signs of drug abuse, chemical contaminants, or biotoxins. System 200 includes a case 210, a test strip 220, and a circuit 240 including a light source 250, a battery 252, a control unit 254, and photodetectors 256 and 258.

Case 210 can be made of plastic or other material suitable for safely containing the liquid sample being analyzed. In the illustrated embodiment, case 210 has an opening through which a portion of test strip 220 extends for application of the sample to a sample-receiving zone 222 of test strip 220. Alternatively, test strip 220 can be enclosed in case 210, for example, when application of the sample to test strip 220 is through an opening in case 210.

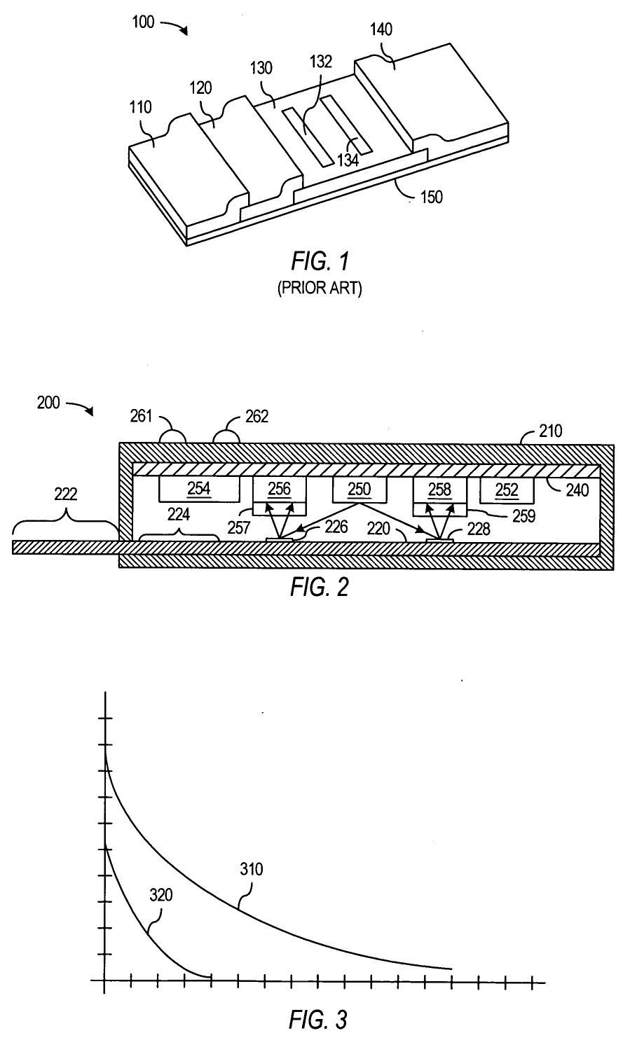

Test strip 220 can be substantially identical to a conventional test strip such as test strip 100 described above in regard to FIG. 1, but in test strip 220, the labeling substance for the target analyte preferably includes a quantum dot or a similar structure that fluoresces at a constant intensity when exposed to light of the proper wavelength. For a test, a user applies a sample to receiving zone 222 of test strip 220. The sample flows from receiving zone 222 into a labeling zone 224 inside case 210. The labeling substance binds the quantum dots or other persistent fluorescent structure to the target analytes. The sample including the labeling substance then enters a capture or detection zone that includes a test stripe 226 and a control stripe 228. Test stripe 226 is a region containing an immobilized substance selected to bind and retain a labeled complex containing the target analyte and the quantum dot. Control stripe 228 is a region containing an immobilized substance selected to bind to and retain to the labeling substance.

Light source 250 in circuit 240 illuminates test stripe 226 and control stripe 228 to cause quantum dots in stripes 226 and 228 to fluoresce. Light source 250 is preferably a light emitting diode (LED) or a laser diode that emits light of a suitable frequency for illumination of test stripe 226 or control stripe 228. Generally, the quantum dots fluoresce under a high frequency (or short wavelength) light, e.g., blue to ultraviolet light, and the fluorescent light has a lower frequency (or a longer wavelength) than the light from light source 250. Test system 200 and particularly test strip 220 generally includes other materials such as nitrocellulose or other organic materials that also fluoresces when exposed to light from light source 250. These materials thus produce background fluorescent light that can complicate precise measurement of the fluorescent light from the quantum dots.

Photodetectors 256 and 258 are in the respective paths of light emitted from test stripe 226 and control stripe 228 and measure the fluorescent light from the respective stripes 226 and 228. A baffle or other light directing structure (not shown) can be used to direct light from test stripe 226 to photodetector 256 and light from control strip 228 to photodetector 258. Photodetectors 256 and 258 optionally have respective color filters 257 and 259 that transmit light of the frequency associated with the fluorescent light from the quantum dots and block other frequencies of light.

In one embodiment of the invention, the labeling substance can include two types of quantum dots. One of the types of quantum dots emits a first wavelength of light and is attached to a substance that binds to the target analyte and to test stripe 226. The other type of quantum dot emits light of a second wavelength and binds to control stripe 228. Color filters 257 and 259 can then be designed so that photodetector 256 measures fluorescent light from the type of quantum dot that test stripe 226 traps when the target analyte is present and photodetector 258 measures fluorescent light from the type of quantum dot that control stripe 228 traps once the flow of liquid has reached control stripe 228.

Quantum dots provide fluorescent light that is generally persistent for a relatively long time after illumination of the quantum dots has stopped. FIG. 3 schematically illustrates a plot 310 of fluorescent light intensity versus time after illumination of a collection of quantum dots has stopped. Plot 320 shows a similar plot 320 of the intensity of fluorescent light from a material such as nitrocellulose. In general, the intensity of fluorescent light from quantum dots or a material drops exponentially with a characteristic half-life. For a typical quantum dot, the half-life for the decay of the fluorescent light is about 25 to 30 ns. However, organic materials such as nitrocellulose typically have a half-life is less than about 10 ns.

FIG. 3 shows that after the illumination is off, the ratio of the intensity of the fluorescent light from quantum dots to the intensity of the fluorescent light from other materials in the test kit generally increases with time because the intensity of fluorescent light from the other materials drops faster than the intensity of the fluorescent light from the quantum dots. This relative persistence of fluorescent light from quantum dots as illustrated in FIG. 3 causes the signal-to-noise ratio (SNR) to improve with time. A gated measurement that measures light intensity after a delay characteristic of the half life of fluorescence from quantum dots can thus improve the sensitivity of a test system such as test system 200 of FIG. 2. Further, electronics implementing a gated measurement with a suitable delay (e.g., 20 to 100 ns) can be constructed at a cost suitable for use in a disposable or semi-disposable structure that is amenable to point of care applications.

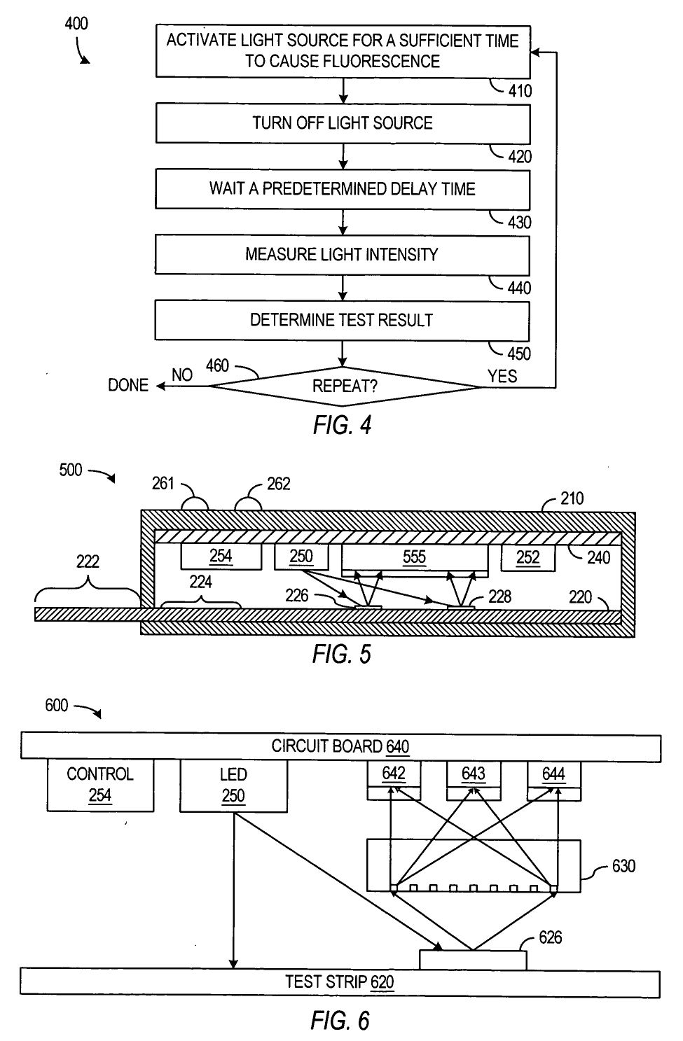

FIG. 4 illustrates an exemplary process 400 using a gated measurement to improve performance of a test system. Process 400 can be implemented in test system 200, for example, in firmware that control unit 254 executes. Process 400 begins in step 410 with activation of a light source. The light source can be left on for any length of time but preferably is on long enough that the fluorescent light from quantum dots in a test stripe reaches maximum intensity. The system then turns off the light source in step 420 and waits a predetermined delay time in step 430 before measuring the light intensity in step 440.

An optimal delay between turning off source (step 420) and measuring the intensity of the fluorescent light from the quantum dots (step 440) will generally depend on all of the sources of noise in the test system. A long delay increases the ratio of fluorescent light from quantum dots to the fluorescent light from other sources as described above, but as the intensity of fluorescent light from quantum dots drops other sources of noise such as light leakage (e.g., into case 210 of system 200) and electronic signal noise become more important. An optimal delay for a specific test kit can be determined that will provide the highest signal-to-noise ratio when accounting for all sources of noise. In a typical embodiment of system 200 including color filters 257 and 259 on photodetectors 256 and 258, a delay time of between about 5 ns and about 100 ns may be optimal. However, delays of up to 200 ns or even up to 500 ns could also be used with quantum dots having longer half-lives for fluorescence.

For system 200, detectors 256 and 258 perform the light intensity measurement (step 440), and the intensity of the fluorescent light from each stripe 256 or 258 is proportional to or otherwise dependent on the number of quantum dots in the corresponding stripe 226 or 228. These intensity measurements thus provide a quantitative indication of the concentration of the target analyte. Step 450 can thus use the intensity measurements to determine a test result that is output from the test system. To implement step 450 in system 200, control unit 254 can be a standard microcontroller or microprocessor with an analog-to-digital converter that receives electrical signals from detectors 256 and 258. The electrical signals from detectors 256 and 258 respectively indicate the measured intensities from stripes 226 and 228 and can be converted to digital values. Control unit 254 can subsequently process the digital measurements and then operate an output system as required to indicate test results.

Optionally, a decision step 460 determines whether the process of steps 410 to 450 is repeated to generate multiple digital measurements of fluorescent light intensity. Processing of the multiple measurements can provide more sensitive/accurate quantitative measurements. One advantage of quantum dots is that a typical quantum dot can be excited and measured more than 10⁶ times per second, allowing gated measurements to be performed at frequencies of about 1 MHz or more. For example, a measurement frequency of about 200 MHz can be achieved when the combined excitation and delay time is 5 ns. In contrast, phosphors having a half life for fluorescent light of about 1 ms or longer can similarly be used with gated measurement to reduce background fluorescence but can only be excited about 100 times per second, assuming each excitation and delayed measurement together take about 100 times the half life of the emitting material. As a result, quantum dots can be much more “luminescent” or show increased sensitivity by factors from about 100 to 10,000 times the sensitivity of a similar system using phosphors.

The output system of system 200 shown in FIG. 2 includes LED lights 261 and 263. Control unit 254 can activate one light 261 when measurements of the fluorescent light from the test stripe 226 indicate the count or concentration of the target analyte in test stripe 226 is above a threshold level. Control unit 254 can activate the other light 262 when the measurements from photodetector 256 indicate that the count or concentration of the target analyte is below the threshold level but the intensity that photodetector 258 measures from control stripe 228 is above a threshold level. A system with three or more LEDs or particular patterns of flashing of one or more LEDs can similarly indicate other test results (e.g., an inconclusive test) or a test status (e.g., to indicate a test in progress).

LED lights 261 and 263 can alternatively be replaced with other types of interfaces. For example, an alphanumeric display can provide a numerical test result based on the measurements of fluorescent light from test stripe 226. Such display could also be used in conjunction with LEDs such as illustrated in FIG. 2 or other output systems. Another test result output technique produces an electric signal via external terminals (not shown) to indicate the test result. An electronic device (not shown) can process, convert, or transmit the test result signal.

FIG. 5 illustrates a test system 500 in accordance with an embodiment of the invention that is similar to system 200 of FIG. 2 but employs an imaging system 555 for detection of fluorescent light from stripes 226 and 228. Imaging system 555 can include a two-dimensional CCD or CMOS imaging array or similar optoelectronic imager capable of generating an electronic representation of an image (e.g., an array of pixel values representing a captured image or frame). The frame rate of imaging system 555 may be limited as described above by the rate at which the quantum dots can be excited or alternatively by the speeds of the electronics. Control unit 254 can analyze one or more digital images that imaging system 455 captured after light source 250 has been shut off for a desired delay. The variation of the intensity and color of light emitted from stripes 226 and 228 can then be used to identify the number of quantum dots in stripe 226 and therefore the desired measurement.

Gated measurements can also be used in test systems employing multiple species of quantum dots. FIG. 6, for example, shows a portion of a test system 600 in accordance with an embodiment of the invention that tests for the presence of multiple target analytes in a sample. Test system 600 includes a test strip 620, an optoelectronic circuit 640, and an intervening optical system 630.

Test strip 620 can be substantially identical to test strip 220, which is described above, but test strip 620 includes multiple labeling substances containing respective species of quantum dots. Each labeling substance binds a corresponding type of quantum dot to a corresponding target analyte. The quantum dots for different labeling substances preferably produce fluorescent light having different characteristic wavelengths (e.g., 525 nm, 595 nm, and 655 nm). Suitable quantum dots having different fluorescent frequencies and biological coatings suitable for binding to analyte-specific immunoglobulins are commercially available from Quantum Dot, Inc. Test strip 620 includes a test stripe 626 that is treated to bind to and immobilize the different complexes including the target analytes and respective labeling substances. Testing for multiple analytes in the same test structure is particularly desirable for cholesterol or cardiac panel test system that measures multiple factors.

Light source 250 illuminates test stripe 626 with light of a wavelength that causes all of the different quantum dots to fluoresce. Fluorescent light from test strip 626 will thus contain fluorescent light of different wavelengths if more than one of the target analytes are present in test strip 626. When light source 250 is turned off, the intensity of fluorescent light falls exponentially as described above, so that after a short delay time, (e.g., about 50 ns to 1 μs) the fluorescent light is almost entirely from the quantum dots.

Optical system 630 separates the different wavelengths of light and focuses each of the different wavelengths on a corresponding photodetector 642, 643, or 644. Photodetectors 642, 643, and 644, which can further include appropriate color filters, thus provide separate electrical signals indicating the number of quantum dots of the respective types in test stripe 626 and therefore indicate concentrations of the respective target analytes. Control circuit 254 can then provide the test results to a user or a separate device as described above.

Optical system 630 in FIG. 6 is an optical substrate providing diffractive focusing of the different wavelengths on different photodetectors 642, 643, and 644. In one embodiment of the invention, optical system 630 includes an optical substrate of a material such as glass or plastic with opaque regions or surface discontinuities in a pattern that provides a desired separation or focusing of the different fluorescent wavelengths. However, diffractive optical elements such as optical system 630 can be fabricated inexpensively using other processes and structures.

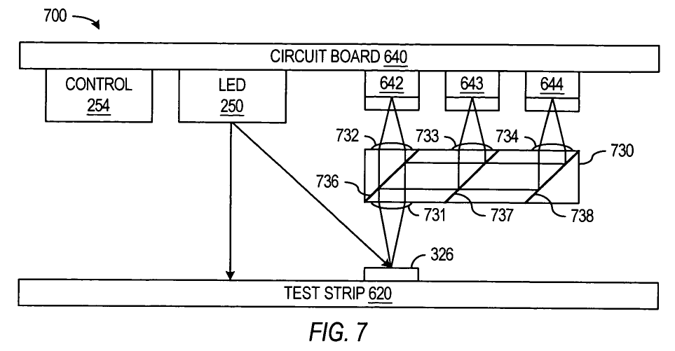

FIG. 7 shows a portion of test system 700 that is similar to test system 600 of FIG. 6, but test system 700 includes an optical system 730 formed from refractive lenses 731, 732, 733, and 734 and thin-film color filters 736, 737, and 738 on prisms. In particular, lens 731 receives and collimates fluorescent light emitted from test stripe 626 when light source 250 illuminates quantum dots in test stripe 626. Color filter 736 transmits light of a frequency corresponding to the quantum dots that photodetector 642 measures and reflects light of the frequency resulting from fluorescence of the other types of quantum dots. Thin films that transmit light of the desired wavelength but reflect light of the other wavelengths can be designed and constructed from a stack of dielectric layers having thicknesses and refractive indices that achieve the desired characteristics. Alternatively, color filter 736 could include a diffractive index grating filter or a colored material. Lens 732 focuses the light transmitted through filter 736 onto the photosensitive area of detector 642, which can include a further color filter for additional selectivity to the desired color of light.

Light reflected from filter 736 is incident on filter 737. Filter 737 is designed to reflect light of the wavelength corresponding to detector 643 and transmit other wavelengths. Lens 733 focuses the light reflected from filter 737 onto the photosensitive area of detector 643. Light transmitted through filter 737 is incident of filter 738, which is designed to reflect light of the wavelength corresponding to detector 644 and transmit the unwanted wavelengths. Lens 734 focuses the light reflected from filter film 738 onto the photosensitive area of detector 644.

Optical systems 630 and 730 merely provide illustrative examples of optical system using diffractive elements or thin-film filters for separating different wavelengths of light for measurements. Optical systems using other techniques (e.g., a chromatic prism) could also be employed to separate or filter the fluorescent light of different frequencies. The characteristics and geometry of such optical systems will generally depend on the number of different types of quantum dots used and the wavelengths of the fluorescent light.

Although the invention has been described with reference to particular embodiments, the description is only an example of the invention's application and should not be taken as a limitation. Various adaptations and combinations of features of the embodiments disclosed are within the scope of the invention as defined by the following claims.

What is claimed is:

1. A rapid diagnostic test system comprising:
a light source for illuminating a medium containing a sample under test, wherein the medium comprises a labeling substance that binds a quantum dot to a target analyte;
a first photodetector positioned to measure light from a test area of the medium; and
a control system coupled to the light source and the photodetector, wherein the control system executes a measurement processes including processing a measurement signal from the photodetector that indicates a light intensity after the light source has been off for a time.

2. The system of claim 1, wherein the light that the first photodetector measures has a frequency characteristic of fluorescent light from the quantum dot.

3. The system of claim 2, wherein the medium comprises a lateral-flow strip for performing a binding assay, and the test area contains an immobilized substance that binds to and holds a complex including the labeling substance and the target analyte.

4. The system of claim 1, wherein the control system processes multiple measurements from the photodetector that indicate the light intensity after the light source has been off for the time.

5. The system of claim 1, further comprising:
a second photodetector; and
an optical system positioned to receive light from the test area, wherein the optical system separates light having a first frequency from light having a second frequency so that the first photodetector measures light having the first frequency and the second photodetector measures light having the second frequency.

6. The system of claim 5, wherein the optical system comprises a diffractive element that directs the light of the first frequency on the first photodetector and directs the light of the second frequency on the second photodetector.

7. The system of claim 5, wherein the optical system comprises a color filter that transmits light having one of the first and second frequencies and reflects light having the other of the first and second frequencies.

8. The system of claim 6, wherein the quantum dot emits fluorescent light having the first frequency; and wherein the medium further comprises a second labeling substance containing a second quantum dot emits fluorescent light having the second frequency.

9. The system of claim 1, wherein the first photodetector comprises a portion of an imaging array that captures an image containing the test area of the medium.

10. The system of claim 1, wherein the first photodetector and the medium are contained in a single-use module.

11. A process for rapid diagnostic testing, comprising:
applying a sample to a medium containing a labeling substance that binds a quantum dot to a target analyte;
illuminating a portion of the medium with light capable of causing the quantum dot to fluoresce;
stopping illumination of the portion of the medium;
measuring light from the portion of the medium after the illumination remains stopped for a delay time; and
determining a test result from the measuring of the light.

12. The process of claim 11, further comprising repeating the illuminating, stopping illumination, and measuring steps a plurality of times, wherein determining the test result uses results from each repetition of measuring the light.

13. The process of claim 12, wherein the measuring step is repeated at a frequency between about 1 MHz and about 200 MHz.

14. The process of claim 11, wherein the medium is in a single-use structure that includes a photodetector that measures light from the quantum dot.

15. The process of claim 1 1, further comprising activating a display on the single-use module to indicate the test result signal.

16. The process of claim 11, further comprising producing an electrical signal that is output from the single-use structure.
